(19) United States
(12) Patent Application Publication
(10) Pub. No.: US 2025/0152613 A1
(43) Pub. Date: May 15, 2025

(54) COMPOSITIONS AND METHODS FOR COVID-19 TREATMENT

(71) Applicant: 101 THERAPEUTICS LTD., TEL AVIV-YAFO (IL)

(72) Inventors: Alec M. GOLDBERG, TEL AVIV-YAFO (IL); Samuel H. GOLDBERG, TEL AVIV-YAFO (IL); James I. GOLDBERG, TEL AVIV-YAFO (IL); Isaiah Z. GOLDBERG, TEL AVIV-YAFO (IL); Michael M. GOLDBERG, TEL AVIV-YAFO (IL)

(73) Assignee: 101 THERAPEUTICS LTD., TEL AVIV-YAFO (IL)

(21) Appl. No.: 18/832,115

(22) PCT Filed: Jan. 24, 2023
(86) PCT No.: PCT/IL2023/050077

§ 371 Date: Jul. 23, 2024

Related U.S. Application Data
(60) Provisional application No. 63/302,575, filed on Jan. 25, 2022.

(51) Int. Cl. A61K 31/704; A61K 45/06; A61P 11/00

(52) U.S. Cl. CPC A61K 31/704; A61K 45/06; A61P 11/00

(57) ABSTRACT

Provided herein are compositions for use in treatment fibrosis and in treatment of COVID-19, preferably moderate COVID-19, comprising Compound (1), or a pharmaceutically acceptable salt thereof. Also provided herein are methods for treatment of COVID-19, preferably moderate COVID-19 comprising administering to a patient in need thereof an amount of between 10 mg/day and 30 mg/day, of Compound (1).

### CROSS REFERENCE TO RELATED APPLICATIONS

Benefit is claimed to U.S. Provisional Patent Application No. 63/302,575 filed Jan. 25, 2022; the contents of which are incorporated by reference herein in its entirety.

### FIELD

This disclosure relates to treatments for COVID-19.

### BACKGROUND

Coronaviruses (CoVs) are members of the order Nidovirales, which includes enveloped viruses with large (˜30 kb), positive-sense single-stranded RNA genomes that yield a characteristic nested set of subgenomic mRNAs during replication in the cytoplasm of infected cells. The genome organization for coronaviruses is highly conserved, with the 5′-most two-thirds of the genome encoding the replicase polyprotein, followed by sequences encoding the canonical structural proteins: spike, envelope, membrane, and nucleocapsid. Many CoVs contain accessory genes, which are interspersed among the genes for the structural proteins. Although these accessory genes are not necessarily required for virus replication and are, in general, not highly conserved within the virus family, many encode proteins that regulate the host response. Interestingly, coronavirus replicase proteins, which are highly conserved, can also act as antagonists to block or delay the host innate immune response to infection. That a slew of coronavirus-encoded accessory and non-accessory proteins have been shown to shape the host antiviral response suggests that viral-mediated subversion of host defenses is an important element of infection.

In the case of the Coronavirus there have been multiple viruses impacting humans including SARS, MERS and SARS-CoV-2.

Severe acute respiratory syndrome coronavirus 2 (SARS-CoV-2), the cause of COVID-19, emerged in China in late 2019. The majority of COVID-19 infections are either asymptomatic or result in mild disease. However, a substantial proportion of older infected individuals do develop a respiratory illness that requires hospitalization. COVID-19 can rapidly progress to critical illness with hypoxemic respiratory failure and require prolonged ventilatory support. The pathophysiology of severe COVID-19 is dominated by an acute pneumonic process with extensive radiological opacity. On autopsy, diffuse alveolar damage, inflammatory infiltrates, and microvascular thrombosis are evident. The host immune response is thought to play a key role in avian influenza, severe acute respiratory syndrome (SARS), and both pandemic and seasonal influenzas. Inflammatory organ injury may occur in severe COVID-19, with a subset of patients having markedly elevated inflammatory markers such as C-reactive protein, ferritin, and interleukins 1 and 6. Several therapeutic interventions to mitigate inflammatory organ injury have been proposed for the treatment of viral pneumonia, but the value of corticosteroids is widely debated. Among COVID-19 patients admitted to UK hospitals, the case fatality rate is over 26%, and in patients requiring invasive mechanical ventilation, the fatality rate is over 37%.

Acute hyperglycemia is regarded as a risk factor for critically ill patients and has been identified as an independent risk factor for adverse outcomes in such patients such as severe infections, multiple organ failure, and death. Acute hyperglycemia has also been found to cause long term damage to insulin secreting islet cells. Patients with Type II diabetes or metabolic syndrome are especially susceptible to severe COVID-19 after infection. Acute hyperglycemia causes difficulty to control glucose in such patients in an intensive care unit setting.

It is known that glucocorticoids significantly increase blood glucose levels in patients. In the RECOVERY trial in which dexamethasone was administered to COVID-19 patients, blood glucose levels were significantly elevated in diabetics (Rayman G, Lumb A N, Kennon B, Cottrell C, Nagi D, Page E, Voigt D, Courtney H C, Atkins H, Higgins K, Platts J, Dhatariya K, Patel M, Newland-Jones P, Narendran P, Kar P, Burr O, Thomas S, Stewart R. Dexamethasone therapy in COVID-19 patients: implications and guidance for the management of blood glucose in people with and without diabetes. Diabet Med. 2021 January; 38 (1): e14378. doi: 10.1111/dme.14378. Epub 2020 Sep. 21). Although dexamethasone may be beneficial in COVID-19, extreme care must be taken when administering to patients in which acute hyperglycemia is especially problematic, such as diabetic patients and patients with metabolic syndrome.

### SUMMARY

Provided herein are compositions for use in treatment of COVID-19, preferably moderate COVID-19, comprising Compound 1, or a pharmaceutically acceptable salt thereof. Compositions for use in treatment of fibrosis are also provided.

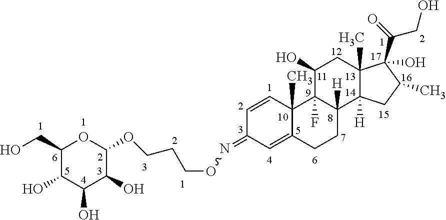

Also provided herein are methods for treatment of COVID-19, preferably moderate COVID-19 comprising administering to a patient in need thereof an amount of between 10 mg/day and 30 mg/day, of Compound 1.

The foregoing and other objects, features, and advantages will become more apparent from the following detailed description, which proceeds with reference to the accompanying figures.

### BRIEF DESCRIPTION OF THE DRAWING

FIG. 1 shows a survival graph of various groups of mice in a bleomycin model of lung fibrosis administered either a compound 1, according to embodiments of the invention early (E) or late (L) versus dexamethasone (DEX), vehicle group or a naïve group.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art. Administration can be local or systemic. Examples of local administration include, but are not limited to, topical administration, intratumoral administration, subcutaneous administration, intramuscular administration, intrathecal administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs (such as the drug delivery devices described herein), which release the active agents and compounds over extended time intervals for sustained treatment effects. An implantable device is “implanted” by any means known to the art of insertion into the tissue or tissue environment that is the area of a given treatment.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Fibrosis: a condition in which fibrous tissue is formed, which may occur as a result of inflammation or damage of tissues in various organs, including but not limited to the lungs. In the lungs, fibrosis is called “pulmonary fibrosis”. COVID-19 may induce pulmonary fibrosis. In addition, other conditions such as cystic fibrosis, idiopathic pulmonary fibrosis, radiation induced lung injury and fibrothorax may be causes of pulmonary fibrosis. Fibrosis may also occur in the liver, heart or kidneys.

Moderate COVID-19: A patient suffering from Moderate COVID-19 has tested positive with a RT-PCR test for SARS-CoV-2 in nasopharyngeal or oropharyngeal swabs; has fever, cough, with or without sore throat/throat irritation, body ache/headache, malaise/weakness, diarrhea or gastrointestinal upset, with or without anorexia/nausea/vomiting, with or without loss of smell and/or taste, shortness of breath/breathlessness and difficulty in breathing; and has a respiratory rate of >24 to <30 breaths/min, SpO₂: 90-93% on room air.

Hyperglycemia: a condition of high glucose in serum, of above 200 mg/dl.

Pharmaceutically Acceptable Salt: The term “pharmaceutically acceptable salt” of a given compound refers to salts that retain the biological effectiveness and properties of the given compound, and which are not biologically or otherwise undesirable. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, methanesulfonic acid, ethanesulfonic acid, p-toluene-sulfonic acid, and the like.

Standard of Care: Accepted treatment as prescribed in guidelines for appropriate treatment for a given indication, based on scientific evidence.

Subject: Living multi-cellular organisms, including vertebrate organisms, a category that includes both human and non-human mammals.

Subject susceptible to a disease or condition: A subject capable of, prone to, or predisposed to developing a disease or condition. It is understood that a subject already having or showing symptoms of a disease or condition is considered “susceptible” since they have already developed it.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound.

### II. Overview of Several Embodiments

Provided herein are methods for treatment of COVID-19 using compound 1 and compositions for use in treatment of COVID-19 comprising compound 1. Also provided herein are methods for treatment of COVID-19, in particular moderate COVID-19, comprising administering a therapeutically effective amount of the compound having the structure:

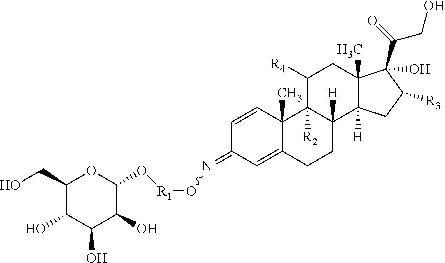

wherein R₁ is a direct bond; or C1-C12 straight alkyl or branched alkyl; R₂ is hydrogen or fluorine; R₃ is a hydrogen or methyl group, and R₄ is a hydroxyl group or a ketone group; and at least one pharmaceutically acceptable carrier. Preferably, the compound or a pharmaceutically acceptable salt thereof, is administered in an amount of between 10 and 30 mg/day. Further aspects of the invention relate to the aforementioned compound for treatment of a disease associated with fibrosis, preferably of the lung, liver, kidney or heart. Optionally, fibrosis of the lung is treated. Optionally, Compound 1 is the agent used in the method for treating disease. Macrophages are white blood cells that are involved in the functioning of the immune system. Macrophages are involved in defending the body from pathogens, wound healing, and immune regulation. There are two main phenotypes of macrophages: M1 macrophages, also known as “classically activated” macrophages and M2 macrophages. M1 macrophages are typically pro-inflammatory, while M2 macrophages are typically anti-inflammatory. Various agents can cause activation of macrophages such as cytokines like interferon-gamma, and bacterial endotoxins, such as lipopolysaccharide. Disturbances in macrophage function can lead to aberrant repair, with uncontrolled inflammatory mediator and growth factor production, deficient generation of anti-inflammatory macrophages, or failed communication between macrophages and epithelial cells, endothelial cells, fibroblasts, and stem or tissue progenitor cells all contributing to a state of persistent injury, which may lead to the development of pathological fibrosis (Immunity. 2016 Mar. 15; 44 (3): 450-462).

Macrophages can be infected by SARS-CoV-2, and their infection is associated with immunoregulatory cytokines secretion and the induction of a macrophagic specific transcriptional program characterized by the upregulation of M2-type molecules (J Infect Dis 2021 Aug. 2; 224 (3): 395-406.) Macrophages, in particular M2 macrophages, play a key role in pathogenesis of fibrosis. (Semin Liver Dis. 2010 August; 30 (3): 245-257.) Suppressing the profibrotic macrophage may provide a rational approach for the treatment or prevention of fibrosis. As opposed to other approaches to treating COVID-19, it is suggested that the compounds described herein are antifibrotic in that they can specifically target M2 macrophages. In clinical settings of fibrotic disease and COVID-19, it is suggested that they ameliorate the pro-fibrotic effects of M2 macrophages.

According to an embodiment, described herein is a pharmaceutical composition comprising a therapeutically effective amount of the compound having the structure:

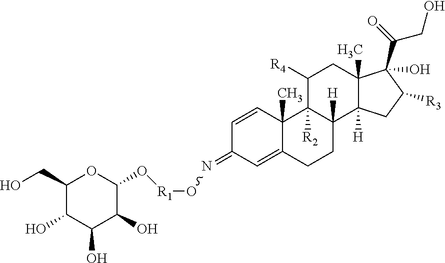

wherein R₁ is a direct bond; or C1-C12 straight alkyl or branched alkyl, optionally substituted with at least one halogen atom selected from fluorine, chlorine and bromine, a hydroxy group, a carboxyl group, an amine group or a benzyl group; R₂ is hydrogen or fluorine; R₃ is a hydrogen or methyl group, and R₄ is a hydroxyl group or a ketone group; and wherein the dotted line represents a single or double bond, and at least one pharmaceutically acceptable carrier. Optionally, R₁ is a non-substituted C1-C10 alkyl group. Optionally, R₁ is a non-substituted (CH₂)₃ group. Optionally, R₂ and R₃ are H and R₄ is a ketone group, and the dotted line is a double bond. Optionally, R₂ is fluorine, R₃ is methyl and R₄ is a hydroxyl group, and the dotted line is a double bond. Optionally, R₂ is H, R₃ is H and R₄ is a hydroxyl group, and the dotted line is a single bond. Optionally, R₂ is H, R₃ is H and R₄ is a ketone group, and the dotted line is a single bond. Optionally, the compound has the structure:

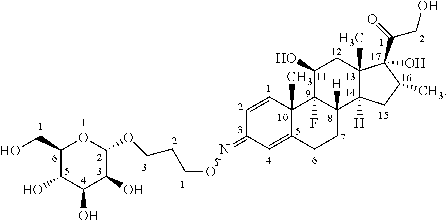

Optionally, the composition is for treatment of fibrosis, optionally, fibrosis of the lung, liver, kidney, heart, muscle, skin, ovaries, or testes, preferably, fibrosis of the lung. Optionally, the composition is for treatment of pulmonary fibrosis associated with COVID-19. Optionally, the compound is administered in an amount of between 10 mg/day and 30 mg/day.

Further described herein is a method for treatment of COVID-19 comprising administering to a patient in need thereof between 10 mg/day and 30 mg/day of a compound 1 having the structure:

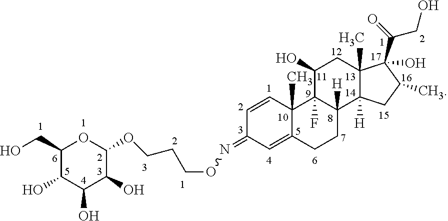

Optionally, the patient is administered 20 mg/day of compound 1. Optionally, the patient is suffering from moderate COVID-19. Optionally, compound 1 is administered for at least three consecutive days. Optionally, the patient's recovery improves relative to treatment with dexamethasone. Optionally, the patient is also receiving a treatment of at least one of: oxygen enrichment, hydration, anti-pyretics, antitussive, multivitamins. antimicrobial agents for co-infections, ivermectin, remdesivir, baricitinib, tofacitinib, tocilizumab, sarilumab. Optionally, compound 1 is administered intravenously. Optionally, the patient does not experience hyperglycemia after administration. Optionally, the patient is suffering from the effects of long COVID-19. Optionally, the patient is further administered standard of care.

Further described herein are methods for treating fibrosis in a patient comprising administering to the patient a therapeutically effective amount of compound 1, optionally, in an amount of between 10 mg/day and 30 mg/day, optionally, 20 mg/day. Optionally, compound 1 is administered intravenously. Optionally, the fibrosis is fibrosis of the lung, liver, kidney, heart, muscle, skin, testes, or ovaries. Optionally, the fibrosis is fibrosis of the lung. Optionally, compound 1 is administered for only 3 days. Optionally, the administration of compound 1 continues at a constant rate, without tapering.

The following examples are provided to illustrate certain particular features and/or embodiments. These examples should not be construed to limit the disclosure to the particular features or embodiments described.

### EXAMPLES

### Example 1: Preparation of Compound 1

Compound 1 was prepared using the following procedure.

### Step 1: (2R,3R,4S,5S,6S)-2-(acetoxymethyl)-6-(3-bromopropoxy)tetrahydro-2H-pyran-3,4,5-triyl triacetate (compound R1)

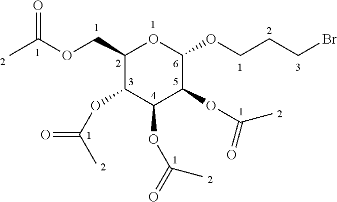 [SMILES: CC(=O)OC[C@H]1O[C@H](OCCCBr)[C@@H](OC(C)=O)[C@@H](OC(C)=O)[C@@H]1OC(C)=O]

1,2,3,4,6-Penta-O-acetyla-D-mannopyranoside (8 gr, 20.5 mmol) was dissolved in CH₂Cl₂ (80 mL) and 3-bromopropan-1-ol (3.13 gr, 22.5 mmol) was added, followed by the addition of boron trifluoride etherate (10.1 mL, 82.0 mmol). The reaction was stirred in the dark under a nitrogen atmosphere for 24 h. TLC analysis (Hexane:EtOAc=1:1) was performed. New spot was detected. DCM was added and the reaction mixture was neutralized by adding saturated NaHCO₃ solution. The phases were separated, the aqueous phase was washed with DCM. The combined organic phase was dried over Na₂SO₄, filtered and evaporated. The crude product was purified by column chromatography (gradient of 3:1 to 1:1-Hexane:EtOAc), the product was isolated as a colorless oil in a yield of 64%.

### Step 2: (2R,3R,4S,5S,6S)-2-(acetoxymethyl)-6-(3-((1,3-dioxoisoindolin-2-yl)oxy) propoxy)tetrahydro-2H-pyran-3,4,5-triyl triacetate (compound R2)

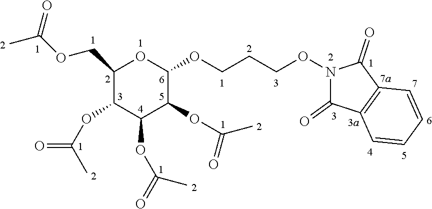 [SMILES: CC(=O)OC[C@H]1O[C@H](OCCCON2C(=O)c3ccccc3C2=O)[C@@H](OC(C)=O)[C@@H](OC(C)=O)[C@@H]1OC(C)=O]

To a solution of compound R₁ (3.5 gr, 7.5 mmol) and N-hydroxylphthalimide (1.35 gr, 8.25 mmol) in DMF (15 mL), DBU (1.1 mL, 8.25 mmol) was added and red solution was stirred for 18 h at room temperature under nitrogen atmosphere. The reaction was monitored by TLC (Hexane/EtOAc=1/1) and LCMS (SB1090), no starting material remained. The yellow-orange solution was added dropwise to the solution of 1N HCl (50 mL). White solid was separated, which was dissolved in EtOAc. The aqueous phase was extracted with EtOAc, dried over Na2SO4, filtered and evaporated. The crude product was purified by silica gel chromatography (gradient of 0-50% EtOAc in Hexane). 3 gr (73% yield) of white solid was obtained.

### Step 3: (2R,3R,4S,5S,6S)-2-(acetoxymethyl)-6-(3-(aminooxy) propoxy)tetrahydro-2H-pyran-3,4,5-triyl triacetate (compound R3)

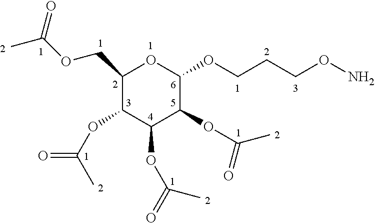 [SMILES: CC(=O)OC[C@H]1O[C@H](OCCCON)[C@@H](OC(C)=O)[C@@H](OC(C)=O)[C@@H]1OC(C)=O]

Compound R2 (3.04 gr, 5.51 mmol) was dissolved in methanol (100 mL) for 30 min due to the hard dissolution and 1.5 eq. of hydrazine hydrate (0.401 mL, 8.27 mmol) was added. The stirring was continued for 4 h, the reaction was monitored by TLC (EtOAc) and LCMS (SB1090). The solvent was removed by evaporation, white solid precipitated (by-product). The crude product was washed with EtOAc, the suspension was filtered, and the mother liquor was evaporated. This washing with EtOAc was repeated 4 times, followed by an additional wash with CHCl₃. The mother liquor was evaporated to dryness. 1.85 gr (80% yield) of colorless oil was obtained. The NMR and LCMS (SB1090) analyses conform the structure.

### Step 4: (2R,3R,4S,5S,6S)-2-(acetoxymethyl)-6-(3-(((8S,9R,10S,11S,13S,14S,16R,17R,E)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-ylidene)amino) propoxy)tetrahydro-2H-pyran-3,4,5-triyl triacetate (compound R4)

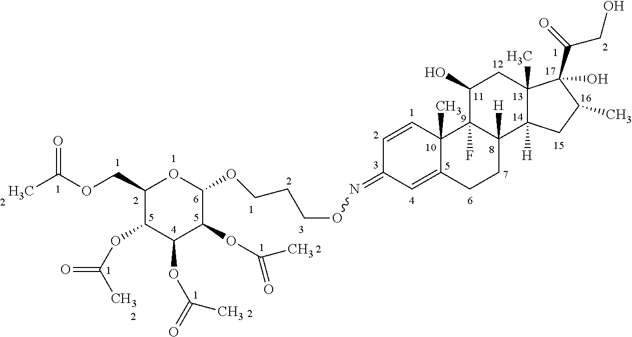

The previously prepared oxime R3 (1.8 gr, 4.27 mmol) was added into a solution of dexamethasone (1.11 gr, 2.85 mmol) in EtOH (25 mL), followed by PTSA (0.27 gr, 1.42 mmol). The reaction mixture was refluxed for 4 h while monitoring by LCMS (SB1090). After 1 h, conversion of 95% was detected. Products of mono/di deprotection of acetate groups were also observed by LCMS. The reaction mixture was cooled to room temperature, NaHCO₃ (1 g) was added and the suspension was stirred for 5 min and filtered. EtOH was evaporated to dryness. Purification by column chromatography (gradient 50% to 100% of EtOAc in Hexane) provided a mixture of the product and partially deprotected by-product. Yield: 1.66 gr. This mixture was used in the next step.

### Step 5: 1-((8S,9R,10S,11S,13S,14S,16R,17R,E)-9-fluoro-11,17-dihydroxy-10,13,16-trimethyl-3-((3-(((2S,3S,4S,5S,6R)-3,4,5-trihydroxy-6-(hydroxymethyl)tetrahydro-2H-pyran-2-yl)oxy) propoxy)imino)-6,7,8,9,10,11,12,13,14,15,16,17-dodecahydro-3H-cyclopenta[a]phenanthren-17-yl)-2-hydroxyethan-1-one (Compound 1)

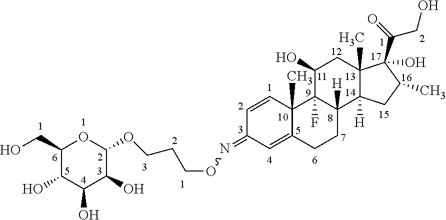

Compound R4 (1.66 gr) was dissolved in 60 mL of MeOH/Et₃N/H₂O (8:1:1). The reaction mixture was stirred at room temperature and monitored by LCMS (SB1090). After 4 h of stirring, only the desired product (m/z 628) was observed. The solvent was evaporated and the product (1.33 g of crude product) was purified by silica gel chromatography, eluted with DCM/MeOH-85/15 and monitored by TLC (DCM/MeOH=80/20). 0.7 gr of white solid was isolated and characterized by NMR and LCMS (SB1090), which confirms the structure.

### Example 2: In Vivo Model of Pulmonary Fibrosis

Post COVID-19 pulmonary fibrosis (PF) is defined as the presence of persistent and different fibrotic tomographic changes, often combined with impairment in pulmonary function tests. The pathogenesis of post-COVID-19 PF is partially known and likely multifactorial. Excessive production of reactive oxygen species and non-protective mechanical ventilation are potential triggers for post-COVID-19 PF. To explore the beneficial effect of compound 1 in pulmonary fibrosis, a bleomycin-induced PF mice model was utilized. Bleomycin is known for its ability to induce lung injury resulting in an acute inflammatory response, and the inflammatory phase is followed by fibrotic changes. In this model, direct administration of bleomycin to the lungs decreases survival of treated mice, due to vast pulmonary damage. The study was performed in 5 treatment groups of C57BL/6 mice (n=10-12 per group) and included early or late treatment with compound 1 (1 mg/mouse), early treatment with dexamethasone (DEX) in an amount of 0.45 mg/mouse, vehicle, and naïve control groups. Early treatment was initiated on day 1, on the same day as bleomycin administration. Late treatment was initiated on day 14. The test items were injected in the vehicle, dexamethasone and Compound 1 early treatment groups, on days 1, 2, 3, 7, 14, 21 and 28. In the late treatment group, Compound 1 was administered on days 14, 15, 16, 21, 28 and 31. The test items were injected intravenously through the tail vein after pulmonary fibrosis model induction (bleomycin was induced by intratracheal administration of bleomycin (3.5 U/kg)). Animals were monitored for morbidity, mortality, body weight, and clinical signs. Selected clinical signs (i.e., physical appearance, activity, ability to respond to external stimulus, eyes, and respiration quality) were scored to provide a clinical score. Cytokine levels in BALF (Day 33) and serum (Days 17 & 32) were determined.

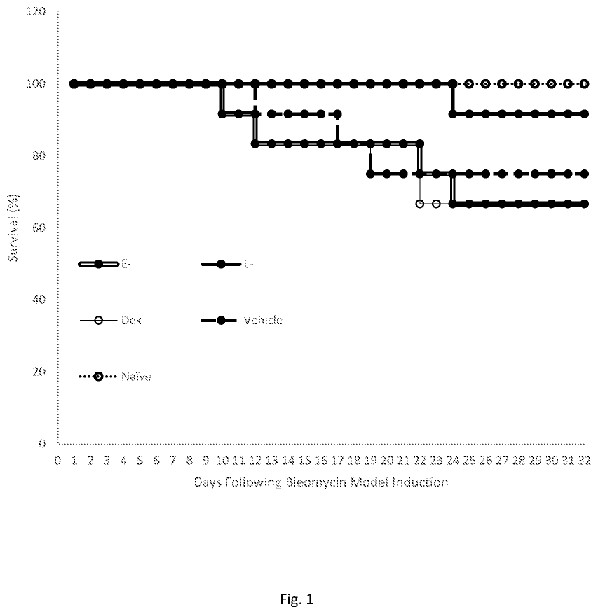

As shown in FIG. 1 similar mortality rate was noted in the vehicle (dashed line), DEX (thin line with hollow marker) and Compound 1 early (designated “E”, double line) treated groups, which was higher than the naïve (dotted line, hollow marker) control group. The Compound 1 late treatment group (Designated “L”, solid line) exhibited lower mortality rate than the DEX and Compound 1 early treated groups and mortality rates similar to the naïve group.

Extensive lung tissue damage is a preliminary step for development of pulmonary fibrosis. Chronic exposure to medications, industrial pollutants, radiation, or acute pulmonary inflammation can induce lung tissue injury and consequent scarring and fibrosis. Histopathological assessment remains as a precise method for evaluation of the severity of pulmonary fibroses. Analysis of the serum and BALF cytokines showed no statistically significant changes in most serum cytokine levels determined on days 17 and 32 among groups (data not shown). Histopathological evaluation (from the end of the study on day 33) revealed reduction in lung fibrosis following the late treatment with Compound 1 and DEX vs. the vehicle control group. The pathological and fibrosis scores and percentage of lung fibrosis by digital morphometry following the late treatment with Compound 1 was slightly lower compared to the DEX treatment, as shown in table 1 below. Values are described in terms of mean±standard deviation.

TABLE 1

| | | Pathological score | Fibrosis score | % Fibrosis - Digital |
|---|---|---|---|---|
| Group | N | (H&E) | (MT) | morphometry |
| Early | 4 | 2.5 ± 0.58 | 2.25 ± 0.5 | 8.25 ± 1.26 |
| comp. 1 | | | | |
| Late | 5 | 2 ± 0.7 | 2 ± 0.7 | 5.2 ± 1.1 |
| comp. 1 | | | | |
| DEX | 4 | 2.25 ± 0.5 | 2.25 ± 0.5 | 6 ± 1.41 |
| Vehicle | 4 | 2.5 ± 0.58 | 3 ± 0.82 | 12.5 ± 8.63 |
| Naïve | 5 | 0.2 ± 0.45 | 0 | 4.2 ± 1.64 |

Histopathological evaluation of H&E and MT-stained lung sections following bleomycin showed the typical pathological characteristics of subacute inflammation and fibrosis throughout the lung tissue. Overall, the late treatment with Compound 1 seems to have a more profound effect in bleomycin lung fibrosis model with respect to mortality, and disease progression as indicated by histopathological evaluation in comparison to early treatment with DEX or Compound 1. This indicates potential of Compound 1 for treatment in cases of moderate to severe COVID-19. Since pulmonary fibrosis has recently emerged as a key complication in the long-term follow-up management of COVID-19, Compound 1, which has been shown to have improved effect in reducing fibrosis over dexamethasone, can be expected to be an effective therapy when administered after pulmonary damage has started.

In addition to a reduction in pulmonary fibrosis, it is suggested that treatment of other types of fibrosis, such as fibrosis of the heart, liver or kidney, may be ameliorated or prevented using Compound 1.

### Example 3: Safety of Compound 1 in Humans

A phase I single-center, open-label, dose-escalation study to assess the safety of Compound 1 in healthy volunteers was initiated. The phase I study included 15 healthy participants divided into single escalating doses in part 1 of the study followed by 3 day repeated doses in part 2 of the study to evaluate the safety of Compound 1 and to identify dose-limiting toxicities (DLTs) before study in COVID-19 patients.

The Phase I study involving 15 subjects, was planned to include single escalating dose part 1 study followed by 3 days repeated dose part 2 study in order to evaluate the safety of Compound 1 in healthy participants and to identify Dose-Limiting Toxicities (DLT) before studied in COVID-19 patients.

Initially, 3 participants were enrolled in a cohort and given a single 10 mg dose of Compound 1 administered intravenously. The second cohort of 3 participants was given a single 20 mg dose of Compound 1 administered intravenously, the third cohort of 6 participants was given a single 30 mg dose of Compound 1 administered intravenously. The dosages of 10, 20 and 30 mg correspond to an equivalent amount of 6.3, 12.5 and 18.8 mg of free dexamethasone, based on the molecular weight calculation of Compound 1, having a molecular weight of 627.31 g/mol and dexamethasone having a molecular weight of 392.464 g/mol.

In the next stage, 30 mg of Compound 1 were given to 3 participants, once a day for 3 consecutive days. A total of 33 Adverse Events (AE) were reported during the study. 4 out of 33 AE were reported before and 29 after the exposer to Investigational Product (IP), 6 out of 29 post exposer AEs were assessed as not related, 13 AEs were possibly related, and 10 AEs are probably related to the IP. Out of 23 possibly or probably related AEs, 6 (26.1%) were hematology AEs, 8 (34.8%) chemistry, 2 (8.7%) insomnia, 2 (8.7%) fatigue reports, 2 (8.7%) skin rash, 1 (4.34%) anxiety 1 (4.34%) hypertension, and 1 (4.34%) noncardiac chest pain. All the events were mild to moderate. All volunteers were discharged on time as planned by the protocol. The phase 1 study results showed no correlation between dose escalation of Compound 1 and AEs frequency or severity. No Serious Adverse Events (SAEs) or severe AEs acquired were detected. No DLTs were reported during the study.

According to the literature, the most frequently reported adverse effect induced by DEX is the presence of insomnia. During Compound 1 administration, 2 cases of insomnia were reported, 1 of anxiety, and 1 of noncardiac chest pain. These events can be explained by discomfort caused by hospitalization or participation in the clinical study; however, a further investigation is required to determine the probability and frequency of these events. A single case of hyperglycemia was detected, the event was mild and transient (Blood Glucose increased from 97 to 105 (mg/dL) in one individual), no changes were seen in the 4 hours post any of the 24 administrations of Compound 1.

### Example 4: Efficacy of Compound 1 in Treatment of COVID-19 in Human Subjects

A prospective, randomized, multicenter, parallel group, double-blind, adaptive Phase II/Phase III study to evaluate safety and efficacy of Compound 1 Vs Dexamethasone for moderate COVID-19 disease along with Standard of Care is performed. The total duration of treatment administration for all patients is 3 days. The endpoints of the study are to evaluate efficacy of Compound 1 versus dexamethasone in moderate COVID-19 patients.

Compound 1 is contained in vials, each vial containing 20 mg of Compound 1 to be reconstituted in saline (0.9% NaCl) for bolus intravenous (IV) injection of 5 ml of solution.

The study is designed to determine rate of treatment-emergent adverse events (AE) and serious adverse events (SAE) including changes in clinical condition, vital signs and laboratory parameters. It is also designed to determine Time to Clinical recovery (TTCR) [Day 1 through Day 10] for which Day of recovery is defined as the first day on which the patient shows 2 points improvement (relative to the patient's highest score) in the 11-point ordinal scale, described in Table 2:

TABLE 2

| | Clinical Status | |
|---|---|---|
| | Ordinal Scale | Clinical Status Description of |
| Patient State | Score | Assessment |
| Uninfected | 0 | Uninfected; no viral RNA detected |
| Ambulatory | 1 | Asymptomatic; viral RNA detected |
| | 2 | Symptomatic; independent |
| | 3 | Symptomatic; assistance needed |
| Hospitalized, | 4 | Hospitalized; no oxygen therapy |
| mild disease | 5 | Hospitalized; oxygen by mask or nasal |
| | | prongs |
| Hospitalized, | 6 | Hospitalized; oxygen by NIVE or high flow |
| severe disease | 7 | Intubation and mechanical ventilation, p |
| | | pO2/FiO2 ≥ 150 or SpO2/FiO2 ≥ 200 |
| | 8 | Mechanical ventilation pO2/FIO2 < 150 |
| | | (SpO2/FiO2 < 200) or vasopressors |
| | 9 | Mechanical ventilation pO2/FiO2 < 150 and |
| | | vasopressors, dialysis, or ECMO |
| Dead | 10 | Death |

Other endpoints of the study include: lowering mortality 28 or 60 days from administration; lowering rate of Intensive care units (ICU) admission; lowering mechanical ventilation or vasopressor therapy (28 days follow-up); improved clinical recovery rate [Time Frame: Day 10]-Proportion of patients who recovered within 10 days for which recovery is defined when the patient improves by 2 points in 11-point ordinal scale; an improvement in temperature, heart rate and oxygen saturation on days 1, 2, 4 and 7; decrease in viral load relative to admission levels according to RT-PCR from nasopharyngeal swab on days 3, 5 and 10; changes in levels of CRP, ferritin and CBC with differential; change in cytokine profile and lymphocyte sub-populations; and measurement of ACTH and morning cortisol levels.

Patients are screened for demographics, physical examination including vital signs, COVID-19 signs and symptoms, medical and surgical history, medication history, RT-PCR, WHO ordinal score and evaluation of Cycle Threshold (CT) value, Urine pregnancy test (only for females with child bearing potential), hematology, biochemistry and urine analysis. Patients with well controlled medical conditions like diabetes, cardiovascular disease including hypertension, CAD, chronic lung/liver or kidney diseases are included in the study based on investigator's judgement.

Approximately 318 eligible patients are randomized in a 1:1 ratio to one of 2 treatment groups. Group 1: Compound 1, 20 mg per day+Standard of care. Group 2: Dexamethasone 6 mg+Standard of care.

Standard of care can include one of, or more than one of: oxygen enrichment, hydration, anti-pyretics, antitussive, multivitamins. antimicrobial agents for co-infections, ivermectin, optionally in a dose of 12 mg per day; remdesivir, baricitinib, tofacitinib, tocilizumab, or sarilumab. Optionally, remdesivir is administered for 5 days, 200 mg IV for the first day, then 100 mg daily for the remaining 4 days.

Total expected study duration is approximately 60 days consisting of: a screening period: 7 days (Day-7 to Day 1) prior to randomization. Treatment period: Day 1 to Day 3. End of treatment assessment: Day 3 (for patients receiving Compound 1) and Day X (for Dexamethasone arm, based on investigator' discretion). Post treatment follow-up: Day 4, 5, 10, 14 and 28. End of study assessment: Day 60. Total study participation duration for respective patients may differ depending on the number of days needed to conduct all screening procedures. Upon screening, the following is performed:

Patient signs the informed consent form; inclusion and exclusion criteria are checked; medical and surgical history, and medication history are recorded; SARS-COV-2 testing (RT-PCR) including both qualitative and quantitative; demography (age, gender) and patient characteristics; urine pregnancy testing (for females of childbearing potential), if applicable; and physical examination.

COVID-19: Signs and Symptoms are determined using the following: laboratory assessments (hematology, biochemistry including C-reactive protein, Ferritin and D-dimer, Cytokines by Multiplex, Sequential Multiple Analysis (SMA), Fasting Plasma Glucose (FPG), Morning Cortisol. T cell subset); urinalysis (routine/microscopic); vital signs (blood pressure, heart rate, respiratory rate and body temperature) and pulse oximetry (oxygen saturation [SpO2]); 12-Lead ECG; Chest X-ray/HRCT (as per investigator's discretion based on his/her clinical judgment); WHO Ordinal 11 point Scale for Clinical Assessment.

On day 1, the patients are enrolled and randomized and treatment begins. The following assessment are performed during days 1-3: COVID-19: signs and symptom assessment; vital signs (blood pressure, heart rate, respiratory rate and body temperature) and pulse oximetry (oxygen saturation [SpO2]); Chest X-ray/HRCT (as per investigator's discretion based on his/her clinical judgment); laboratory assessments (hematology, biochemistry including Cytokines by Multiplex, Sequential Multiple Analysis (SMA), Fasting Plasma Glucose (FPG)); urinalysis (routine/microscopic); WHO 11 point Ordinal Scale for Clinical Assessment.

On days 5 and 10, similar assessments are performed. Adverse events and serious adverse events are recorded. In days 14, 28 and 60, vital signs and WHO 11 point ordinal scale for clinical assessment is performed.

It is expected that recovery from moderate COVID-19 will be enhanced in the group to which Compound 1 is administered relative to the dexamethasone group. It is also expected that hyperglycemia will be lower in the Compound 1 group relative to the dexamethasone group. It is expected that Compound 1 group will show enhanced parameters of lower mortality.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treatment of fibrosis, comprising administering to a patient in need thereof a composition comprising a therapeutically effective amount of the compound having the structure: 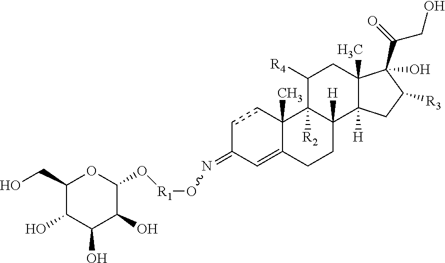
wherein R₁ is a direct bond; or C1-C12 straight alkyl or branched alkyl, optionally substituted with at least one halogen atom selected from fluorine, chlorine and bromine, a hydroxy group, a carboxyl group, an amine group or a benzyl group; R₂ is hydrogen or fluorine; R₃ is a hydrogen or methyl group, and R₄ is a hydroxyl group or a ketone group; and wherein the dotted line represents a single or double bond, and at least one pharmaceutically acceptable carrier.

2. The method according to claim 1 wherein R₁ is a non-substituted C1-C10 alkyl group.

3. The method according to claim 2 wherein R₁ is a non-substituted (CH₂)₃ group.

4. The method according to claim 3 wherein R₂ and R₃ are H and R₄ is a ketone group, and the dotted line is a double bond.

5. The method according to claim 3 wherein R₂ is fluorine, R₃ is methyl and R₄ is a hydroxyl group, and the dotted line is a double bond.

6. The method according to claim 3 wherein R₂ is H, R₃ is H and R₄ is a hydroxyl group, and the dotted line is a single bond.

7. The method according to claim 3 wherein R₂ is H, R₃ is H and R₄ is a ketone group, and the dotted line is a single bond.

8. The method according to claim 1, wherein the compound has the structure:

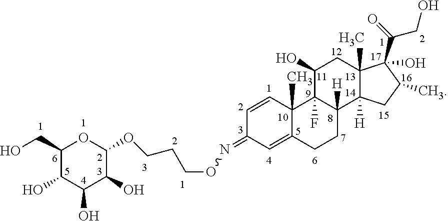

9. The method according to claim 1, wherein fibrosis is fibrosis of the lung, liver, kidney, heart, muscle, skin, ovaries, or testes.

10. The method according to claim 9 wherein fibrosis is fibrosis of the lung.

11. The method according to claim 1 for treatment of pulmonary fibrosis associated with COVID-19.

12. The method according to claim 1, wherein the compound is administered in an amount of between 10 mg/day and 30 mg/day.

13. A method for treatment of COVID-19 comprising administering to a patient in need thereof between 10 mg/day and 30 mg/day of a compound 1 having the structure:

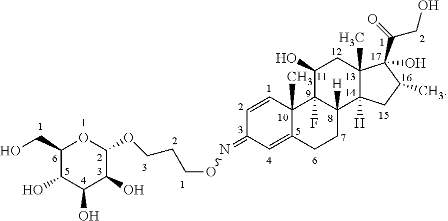

14. The method according to claim 13 wherein the patient is administered 20 mg/day of compound 1.

15. The method according to claim wherein the patient is suffering from moderate COVID-19.

16. The method according to claim 13 wherein compound 1 is administered for at least three consecutive days.

17. The method according to claim 13 wherein the patient's recovery improves relative to treatment with dexamethasone.

18. The method according to claim 13 wherein the patient is also receiving a treatment of at least one of: oxygen enrichment, hydration, anti-pyretics, antitussive, multivitamins, antimicrobial agents for co-infections, ivermectin, remdesivir, baricitinib, tofacitinib, tocilizumab, sarilumab.

19. (canceled)

20. (canceled)

21. The method according to claim 13 wherein the patient is suffering from the effects of long COVID-19.

22. The method according to claim 13 wherein the patient is further administered standard of care.

23.-41. (canceled)
